(19) United States
(12) Patent Application Publication
(10) Pub. No.: US 2015/0004633 A1
(43) Pub. Date: Jan. 1, 2015

(54) ASSAYS AND METHODS FOR THE DIAGNOSIS OF OVARIAN CANCER

(71) Applicant: Quest Diagnostics Investments Incorporated, Wilmington, DE (US)

(72) Inventors: Kenneth Sisco, Chantilly, VA (US); Peter Chou, Chantilly, VA (US)

(73) Assignee: Quest Diagnostics Investments Incorporated, Wilmington, DE (US)

(21) Appl. No.: 14/377,069

(22) PCT Filed: Sep. 26, 2012
(86) PCT No.: PCT/US12/57315

§ 371 Date: Aug. 6, 2014

Related U.S. Application Data
(60) Provisional application No. 61/596,107, filed on Feb. 7, 2012.

(51) Int. Cl. G01N 33/574; G06F 19/00; G06F 17/18; G06F 19/24

(52) U.S. Cl. CPC G01N 33/57449; G06F 19/24; G06F 19/3431; G06F 17/18; G01N 2333/5412
USPC 435/7.92; 205/792; 702/19

(57) ABSTRACT

Provided are methods for diagnosing ovarian cancer or assessing the risk of developing ovarian cancer in a subject by measuring, in a biological sample from the subject, the amount of IL-6 and comparing the amount of IL-6 measured to a predetermined IL-6 cutoff value. Also provided are methods that further include measuring, in the biological sample, the amount of two or more biomarkers selected from the group consisting of transthyretin, apolipoprotein A1, transferrin, β-2 microglobulin, and CA 125 II. The amount of IL-6 and biomarkers are useful in the diagnosis of ovarian cancer, and individuals can be identified as having ovarian cancer when the amount of IL-6 is greater than the IL-6 cutoff value and/or the biomarker score is greater than the biomarker score cutoff value.

### FIELD OF THE INVENTION

The invention relates to medically useful assays and methods for the diagnosis of ovarian cancer.

### BACKGROUND OF THE INVENTION

Ovarian cancer is among the most lethal gynecologic malignancies in developed countries. Annually, in the United States alone, approximately 23,000 women are diagnosed with the disease and almost 14,000 women die from it. (Jamal et al., CA Cancer J. Clin., 52:23-47 (2002)). Despite progress in cancer therapy, ovarian cancer mortality has remained virtually unchanged over the past two decades. Given the steep survival gradient relative to the stage at which the disease is diagnosed, early detection remains the most important factor in improving long-term survival of ovarian cancer patients.

The identification of tumor markers suitable for the early detection and diagnosis of cancer holds great promise to improve the clinical outcome of patients. It is especially important for patients presenting with vague or no symptoms or with tumors that are relatively inaccessible to physical examination. As more tumor biomarkers are discovered, tests can be modified to provide increased sensitivity and specificity based on the detection of such tumor markers.

The poor prognosis of ovarian cancer diagnosed at late stages, the cost and risk associated with confirmatory diagnostic procedures, and its relatively low prevalence in the general population together pose extremely stringent requirements on the sensitivity and specificity of a test for it to be used for screening for ovarian cancer in the general population.

Thus, it is desirable to have a reliable and accurate method of determining the ovarian cancer status in patients, the results of which can then be used to manage patient treatment.

### SUMMARY OF THE INVENTION

The instant invention is based on the discovery that interleukin 6 (IL-6) can be used as a biomarker to diagnose ovarian cancer or to assess the risk (i.e., the likelihood) of an individual to develop ovarian cancer.

In one aspect, the invention provides a method for diagnosing or identifying the risk of an individual for having ovarian cancer comprising: (a) measuring, in a biological sample from the subject, the amount of IL-6; (b) comparing the amount of IL-6 measured in step (a) to a predetermined IL-6 cutoff value; and (c) identifying the individual as identifying the individual as being at risk for having (or having) ovarian cancer when the amount of IL-6 is greater than the IL-6 cutoff value, and identifying the individual as not at risk for having (or having) ovarian cancer when the amount of IL-6 is less than the IL-6 cutoff value.

The term “subject,” refer to a patient, e.g., female human, who want to establish ovarian cancer status. The subjects may be women who have been determined to have a high risk of ovarian cancer based on their family history. Other patients include women who have ovarian cancer and the test is being used to determine the effectiveness of therapy or treatment they are receiving. Also, patients may include healthy women who are having a test as part of a routine examination, or to establish baseline levels of the biomarkers. Samples may be collected from women who have been diagnosed with ovarian cancer and received treatment to eliminate the cancer, or perhaps are in remission. In one embodiment, the subject is a post-menopausal woman. In another embodiment, the subject is a pre-menopausal woman.

IL-6 refers to a protein or DNA sequence encoded by the IL-6 gene. The following NCBI accession numbers are associated with human IL-6 protein sequence: P05231.1, NP 000591.1 and AAH15511.1. The NCBI accession number NM 000600.3 describes the human mRNA sequence of this gene. Each of these NCBI accession number references and the sequence associated with each accession number is herein incorporated by reference in its entirety.

The term “cutoff value” refers to a predetermined numerical value that describes the value that demarcates the line between two different diagnoses. For example, in ovarian cancer, the IL-6 cutoff value can be a numerical value in which any value determined above such cutoff is considered to be derived from a patient considered as being at risk or, alternatively, being at increased risk for having ovarian cancer and any value determined below such cutoff is considered to be derived from a patient considered as not being at risk or, alternatively, being at low risk for having ovarian cancer. In one embodiment, determined values above the cutoff value indicate a diagnosis of malignant ovarian cancer and determined values below the cutoff value indicate no malignant ovarian cancer and/or benign tumors. The cutoff value may have units or be unit less. In one embodiment, the predetermined cutoff value is derived from a measurement of the amount of IL-6 in one or more subjects that do not have ovarian cancer. In a further embodiment, the IL-6 cutoff value is about 5 pg/mL Alternatively, the IL-6 cutoff value is about 3.5 pg/mL, about 4 pg/mL, about 4.5 pg/mL, about 5.5 pg/mL, about 6 pg/mL, about 6.5 pg/mL, about 7 pg/mL, about 8 pg/mL, about 8.1 pg/mL, or about 8.5 pg/mL. The cutoff value may be determined experimentally or mathematically. Such methods for determining a cutoff value experimentally and mathematically are described herein.

In one embodiment, the method further comprises (i) measuring, in the biological sample, the amount of a biomarker selected from the group consisting of transthyretin, apolipoprotein A1, transferrin, β-2 microglobulin, and CA 125 II, (ii) comparing the amount of the biomarker measured in step (i) to a predetermined biomarker cutoff value; and (iii) identifying the individual as being at risk for having ovarian cancer when the amount of IL-6 is greater than the IL-6 cutoff value and the amount of the biomarker is greater than the biomarker cutoff value, and identifying the identifying the individual as not at risk for having ovarian cancer when either or both of the amount of IL-6 is less than the IL-6 cutoff value and the amount of the biomarker is less than the biomarker cutoff value.

In another embodiment, the method further comprises: (i) measuring, in the biological sample, the amount of two or more biomarkers selected from the group consisting of transthyretin, apolipoprotein A1, transferrin, β-2 microglobulin, and CA 125 II, (ii) calculating a biomarker score from the results of step (i); (iii) comparing the biomarker score to a predetermined biomarker score cutoff value; and (iv) identifying the individual as being at risk for having ovarian cancer when the amount of IL-6 is greater than the IL-6 cutoff value and the biomarker score is greater than the biomarker score cutoff value, and identifying the individual as not at risk for having ovarian cancer when either or both of the amount of IL-6 is less than the IL-6 cutoff value and the biomarker score is less than the biomarker score cutoff value. In one embodiment, the amount of three or more biomarkers selected from the group consisting of transthyretin, apolipoprotein A1, transferrin, β-2 microglobulin, and CA 125 II are measured and used to calculate the biomarker score. In another embodiment, he amount of four or more biomarkers selected from the group consisting of transthyretin, apolipoprotein A1, transferrin, β-2 microglobulin, and CA 125 II are measured and used to calculate the biomarker score. In one embodiment, the amount of transthyretin, apolipoprotein A1, transferrin, β-2 microglobulin, and CA 125 II are measured and used to calculate the biomarker score. In a related embodiment, the biomarker score for transthyretin, apolipoprotein A1, transferrin, β-2 microglobulin, and CA 125 II is determined from an OVA1 test. In a further embodiment, the biomarker score cutoff value is about 5, or alternatively, about 4, or about 4.5, or about 5.5, or about 6, or about 6.5, or about 7, or about 7.5, or about 8, or about 8.5. In one embodiment, the biomarker score cutoff value is about 8.1 and the IL-6 cutoff value is 5.0 pg/mL.

The terms “transthyretin” or “TTR” refers to a protein or DNA sequence encoded by the TTR gene. The following NCBI accession numbers are associated with human TTR protein sequence: AAD14937.2, P02766.1, AAB36045.1, AAD14098.1, ABI63351.1, ABI63345.1, CAA42087.1, NP_{—}000362.1 and AAD45014.1. The NCBI accession number NM_{—}000371.3 describes the human mRNA sequence of this gene. Each of these NCBI accession number references and the sequence associated with each accession number is herein incorporated by reference in its entirety.

The terms “apolipoprotein A1” or “ApoA1” refers to a protein or DNA sequence encoded by the APOA1 gene. The following NCBI accession numbers are associated with the human ApoA1 protein sequence: CAA00975.1, P02647.1, NP_{—}000030.1, AAS68227.1, ACA05936.1, ACA05935.1, ACA05934.1, ACA05933.1 and ACA05932.1. The NCBI accession number NM_{—}000039.1 describes the human mRNA sequence of this gene. Each of these NCBI accession number references and the sequence associated with each accession number is herein incorporated by reference in its entirety.

The terms “transferrin” or “TF” refers to a protein or DNA sequence encoded by the TF gene. The following NCBI accession numbers are associated with the human transferrin protein sequence: NP_{—}001054, NP_{—}054830, AAB22049.1, AAB97880.1, AAA61141.1, and ABI97197.1. The NCBI accession numbers NM_{—}001063.3 and NM_{—}014111 describe the human mRNA sequence of this gene. Each of these NCBI accession number references and the sequence associated with each accession number is herein incorporated by reference in its entirety.

The terms “β-2 microglobulin” or “B2M” refers to a protein or DNA sequence encoded by the B2M gene. The following NCBI accession numbers are associated with the human B2M sequence: NP_{—}004039.1 (protein) and NM_{—}004048.2 (mRNA). Each of these NCBI accession number references and the sequence associated with each accession number is herein incorporated by reference in its entirety.

The terms “CA 125 II” “mucin 16” or “MUC16” refer to a protein that in humans is encoded by the MUC16 gene. The following NCBI accession numbers are associated with the human MUC16 protein sequence NP_{—}078966.2 (protein) and NM_{—}024690.2 (mRNA). Each of these NCBI accession number references and the sequence associated with each accession number is herein incorporated by reference in its entirety.

The term “diagnose” as used herein refers to the act or process of identifying or determining a disease or condition in a mammal or the cause of a disease or condition by the evaluation of the signs and symptoms of the disease or disorder. Usually, a diagnosis of a disease or disorder is based on the evaluation of one or more clinical factors and/or symptoms that are indicative of the disease. That is, a diagnosis can be made based on the presence, absence or amount of a factor which is indicative of presence or absence of the disease or condition. Each factor or symptom that is considered to be indicative for the diagnosis of a particular disease does not need be exclusively related to the particular disease; i.e. there may be differential diagnoses that can be inferred from a diagnostic factor or symptom. Likewise, there may be instances where a factor or symptom that is indicative of a particular disease is present in an individual that does not have the particular disease.

All numerical designations, e.g., pH, temperature, time, concentration, and molecular weight, including ranges, are approximations which are varied (+) or (−) by increments of 1.0 or 0.1, as appropriate or alternatively by a variation of +/−15%, or alternatively 10% or alternatively 5% or alternatively 2%. It is to be understood, although not always explicitly stated, that all numerical designations are preceded by the term “about”. It also is to be understood, although not always explicitly stated, that the reagents described herein are merely exemplary and that equivalents of such are known in the art.

As used in the specification and claims, the singular form “a”, “an” and “the” include plural references unless the context clearly dictates otherwise. For example, the term “a polypeptide” includes a plurality of polypeptides, including mixtures thereof.

As used herein, the term “comprising” is intended to mean that the compositions and methods include the recited elements, but do not exclude others. “Consisting essentially of” when used to define compositions and methods, shall mean excluding other elements of any essential significance to the combination for the intended use. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions of this invention. Embodiments defined by each of these transition terms are within the scope of this invention.

Unless defined otherwise, all technical and scientific terms used herein have the same meanings as commonly understood by one of ordinary skill in the art to which this invention belongs.

### DETAILED DESCRIPTION OF THE INVENTION

This invention is predicated on the finding that determining the amount of IL-6, either alone or in combination with other biomarkers, can be used to diagnose ovarian cancer.

Sample Preparation

Provided herein are methods of using the information obtained through analysis of the amount of certain biomarkers in biological samples of acellular biological sample or cell-containing samples. Test samples may be obtained from an individual or patient. Methods of obtaining test samples are well-known to those of skill in the art and include, but are not limited to, aspirations or drawing of blood or other fluids. Samples may include, but are not limited to, whole blood, serum, plasma, saliva, urine, and amniotic fluid. In one embodiment, the biological sample is serum or plasma.

In embodiments in which the amount of the biomarker will be determined using an acellular body fluid, the test sample obtained from a person may be a cell-containing liquid or an acellular body fluid (e.g., plasma or serum). In some embodiments in which the test sample contains cells, the cells may be removed from the liquid portion of the sample by methods known in the art (e.g., centrifugation) to yield acellular body fluid for the determination of the amount of certain biomarkers described herein.

In other embodiments, the amount of the biomarker can be determined using a cell-containing sample. In these embodiments the cell-containing sample includes, but is not limited to, blood, urine, organ, and tissue samples (e.g., biopsy). Cell lysis may be accomplished by standard procedures. In certain preferred embodiments, the cell-containing sample is a whole blood cell lysate. In certain other embodiments, the cell-containing sample is a white blood cell lysate. Methods for obtaining white blood cells from blood are known in the art (Rickwood et al., Anal. Biochem. 123:23-31 (1982); Fotino et al., Ann. Clin. Lab. Sci. 1:131 (1971)). Commercial products useful for cell separation include without limitation Ficoll-Paque (Pharmacia Biotech) and NycoPrep (Nycomed).

Measuring Biomarkers in a Biological Sample

Aspects of this invention relate to the detection and quantification of certain biomarkers in a sample. Suitable biophysical or biomolecular detection methods for qualitatively and quantitatively detecting a biomarker comprise any suitable method known in the art. Such methods include, without being limited thereto, methods as applied for qualitative or quantitative assays such as, for example, electrochemical methods (voltametry and amperometry techniques), atomic force microscopy, radio frequency methods, e.g., multipolar resonance spectroscopy, Enzyme-linked Immunosorbent Assay (ELISA), ELISPOT-Assay, Mass spectrometry, Western-Blot or Immunoassays. Such methods may comprise optical, radioactive, chromatographic methods, fluorescence detection methods, radioactivity detection methods, Coomassie-Blue staining, Silver staining or other protein staining methods, electron microscopy methods, methods for staining tissue sections by immunohistochemistry or by direct or indirect immunofluorescence, etc. Also included are methods that measure the amount of biomarker by measuring the amount of DNA. Such methods include real-time PCR, reverse transcriptase-PCR, Southern blot, and the like.

Immunoassays, such as an ELISA are commonly used for the detection of biomarkers in a biological sample. In one example of an ELISA, the antibodies specific for the biomarker are immobilized on a selected surface, such as a well in a polystyrene microtiter plate, dipstick, or column support. Then, a test composition suspected of containing the desired biomarker, such as a biological sample, is added to the wells. After binding and washing to remove non specifically bound immune complexes, the bound biomarker may be detected. Detection is generally achieved by the addition of another antibody, specific for the desired biomarker that is linked to a detectable label. This type of ELISA is known as a “sandwich ELISA.” Detection also may be achieved by the addition of a second antibody specific for the desired biomarker, followed by the addition of a third antibody that has binding affinity for the second antibody, with the third antibody being linked to a detectable label. Variations on ELISA techniques are known to those of skill in the art. In one embodiment, the amount of IL-6 is determined using an ELISA assay. In a related embodiment, the ELISA assay used to determine the IL-6 is a sandwich ELISA assay.

As used herein, the term “label” intends a directly or indirectly detectable compound or composition that is conjugated directly or indirectly to the composition to be detected, for example, N-terminal histadine tags (N-His), magnetically active isotopes, e.g., ¹¹⁵Sn, ¹¹⁷Sn and ¹¹⁹Sn, a non-radioactive isotopes such as ¹³C and ¹⁵N, polynucleotide or protein such as an antibody so as to generate a “labeled” composition. The term also includes sequences conjugated to the polynucleotide that will provide a signal upon expression of the inserted sequences, such as green fluorescent protein (GFP) and the like. The label may be detectable by itself (e.g. radioisotope labels or fluorescent labels) or, in the case of an enzymatic label, may catalyze chemical alteration of a substrate compound or composition which is detectable. The labels can be suitable for small scale detection or more suitable for high-throughput screening. As such, suitable labels include, but are not limited to magnetically active isotopes, non-radioactive isotopes, radioisotopes, fluorochromes, chemiluminescent compounds, dyes, and proteins, including enzymes. The label may be simply detected or it may be quantified. A response that is simply detected generally comprises a response whose existence merely is confirmed, whereas a response that is quantified generally comprises a response having a quantifiable (e.g., numerically reportable) value such as an intensity, polarization, and/or other property. In luminescence or fluorescence assays, the detectable response may be generated directly using a luminophore or fluorophore associated with an assay component actually involved in binding, or indirectly using a luminophore or fluorophore associated with another (e.g., reporter or indicator) component. Examples of luminescent labels that produce signals include, but are not limited to bioluminescence and chemiluminescence. Detectable luminescence response generally comprises a change in, or an occurrence of, a luminescence signal. Suitable methods and luminophores for luminescently labeling assay components are known in the art and described in, for example, Haugland, Richard P. (1996) Handbook of Fluorescent Probes and Research Chemicals (6^{th} ed.). Examples of luminescent probes include, but are not limited to, aequorin and luciferases.

Competition ELISAs are assays in which test samples compete for binding with known amounts of labeled proteins. The amount of reactive species in the unknown sample is determined by mixing the sample with the known labeled species before or during incubation with coated wells. The presence of reactive species in the sample acts to reduce the amount of labeled species available for binding to the well and thus reduces the ultimate signal. Irrespective of the format employed, ELISAs have certain features in common, such as coating, incubating or binding, washing to remove non specifically bound species, and detecting the bound immune complexes.

Antibodies may also be linked to a solid support, such as in the form of plate, beads, dipstick, membrane, or column matrix, and the sample to be analyzed is applied to the immobilized antigen or antibody. In coating a plate with either antigen or antibody, one will generally incubate the wells of the plate with a solution of antibody, either overnight or for a specified period. The wells of the plate will then be washed to remove incompletely-adsorbed material. Any remaining available surfaces of the wells are then “coated” with a nonspecific protein that is antigenically neutral with regard to the test antisera. These include bovine serum albumin (BSA), casein, and solutions of milk powder. The coating allows for blocking of nonspecific adsorption sites on the immobilizing surface and thus reduces the background caused by nonspecific binding of antisera onto the surface.

In the method of the present invention for detecting the presence of at least one biomarker in a sample a quantitative determination can be carried out. “Quantitative determination” in the context of the inventive method is to be understood as any method for determination of an antibody or proteins or peptides, protein fragments, variants or epitopes thereof, known by a skilled person suitable for quantifying the amount of a autoantibody or a secondary antibody, in a sample. As an example, the inventive method may be carried out with a test sample as a concurrent standard, containing a defined amount of a biomarker, and in parallel with a second sample, which is derived from a patient and contains an unknown amount of a biomarker to be determined against. A comparison of the defined amount of the biomarker in the test sample with the amount of the biomarker in the second sample will allow a precise determination of the amount of biomarker in the second sample. A concurrent standard may be applied either parallel to carrying out the inventive method or, for example, prior to said method, by preparing a standard curve, which may be used in the subsequent quantification.

In one embodiment, the amount of IL-6 and the one or more biomarkers are measured by one or more methods selected from the group consisting of immunonephelometry, electrochemiluminescence, and ELISA. Immunonephelometry is a technique used to determine levels of antibodies or antibody/antigen complexes in a sample. It is performed by measuring the turbidity in a water sample by passing light through the sample being measured. In immunonephelometry the measurement is made by measuring the light passed through a sample at an angle. This technique is widely used in clinical laboratories because it is relatively easily automated. It is based on the principle that a dilute suspension of small particles will scatter light (usually a laser) passed through it rather than simply absorbing it. The amount of scatter is determined by collecting the light at an angle (usually at 30 and 90 degrees). Antibody and the antigen (e.g. biomarker) are mixed in concentrations such that only small aggregates are formed that do not quickly settle to the bottom. The amount of light scatter is measured and compared to the amount of scatter from known mixtures. The amount of the unknown is determined from a standard curve.

Immunonephelometry is typically performed with antibody as the reagent and the patient antigen (or biomarker) as the unknown. In the Immunology Medical Lab, two types of tests can be run: “end point immunonephelometry” and “kinetic (rate) immunonephelometry”. End point immunonephelometry tests are run by allowing the antibody/antigen reaction to run through to completion (until all of the present reagent antibodies and the present patient sample antigens that can aggregate have done so and no more complexes can form). Unfortunately, the large particles will fall out of the solution and cause a false scatter reading, thus kinetic immunonephelometry was devised. In kinetic immunonephelometry, the rate of scatter is measured right after the reagent is added. As long as the reagent is constant the rate of change can be seen as directly related to the amount of antigen or biomarker present.

In yet another embodiment, the method includes determining the amount of the biomarker by electrochemiluminesence. An electrochemiluminesence immunoassay “ECLIA” is an assay in which a biomarker bound to labeled antibodies is coupled to microparticles. The microparticles are magnetically captured onto the surface of the electrode. Application of a voltage to the electrode induces a chemiluminescent emission which is measured by a photomultiplier. In one embodiment, CA 125 II is measured by the method of electrochemiluminescence.

Determining the Cutoff Value

The biomarkers of the invention can be used in diagnostic tests to indicate whether a patient is at risk or has a high risk of having ovarian cancer. Such methods can be useful for diagnosing the risk of or increased risk of ovarian cancer. Such diagnoses can include, for example, risk of or a high risk of disease (e.g., ovarian cancer (malignant) versus ovarian cancer of low malignant potential versus benign ovarian disease versus other malignant conditions), the risk of developing disease, the stage of the disease, the progress of disease (e.g., progress of disease or remission of disease over time) and the effectiveness or response to treatment of disease. Based on this diagnosis, further procedures may be indicated, including additional diagnostic tests or therapeutic procedures or regimens.

The correlation of test results with ovarian cancer status can be done by applying a classification algorithm of some kind to the results to generate the status. The classification algorithm may be as simple as determining whether or not the amount of a given biomarker measured is above or below a particular cutoff number. When multiple biomarkers are used, the classification algorithm may be a linear regression formula. Alternatively, the classification algorithm may be the product of any of a number of learning algorithms.

Classification models can be formed using any suitable statistical classification (or “learning”) method that attempts to segregate bodies of data into classes based on objective parameters present in the data. Classification methods may be either supervised or unsupervised. Examples of supervised and unsupervised classification processes are described in Jain, “Statistical Pattern Recognition: A Review,” IEEE Transactions on Pattern Analysis and Machine Intelligence, Vol. 22, No. 1, January 2000, the teachings of which are incorporated by reference.

In supervised classification, training data containing examples of known categories are presented to a learning mechanism, which learns one or more sets of relationships that define each of the known classes. New data may then be applied to the learning mechanism, which then classifies the new data using the learned relationships. Examples of supervised classification processes include linear regression processes (e.g., multiple linear regression (LR), partial least squares (PLS) regression and principal components regression (PCR)), binary decision trees (e.g., recursive partitioning processes such as CART--classification and regression trees), artificial neural networks such as back propagation networks, discriminant analyses (e.g., Bayesian classifier or Fischer analysis), logistic classifiers, and support vector classifiers (support vector machines).

One supervised classification method is a recursive partitioning process. Recursive partitioning processes use recursive partitioning trees to classify data derived from unknown samples. Further details about recursive partitioning processes are provided in U.S. patent application No. 2002/0138208 A1 to Paulse et al., “Method for Analyzing Mass Spectra.”

In other embodiments, the classification models that are created can be formed using unsupervised learning methods. Unsupervised classification attempts to learn classifications based on similarities in the training data set, without pre-classifying the spectra from which the training data set was derived. Unsupervised learning methods include cluster analyses. A cluster analysis attempts to divide the data into “clusters” or groups that ideally should have members that are very similar to each other, and very dissimilar to members of other clusters. Similarity is then measured using some distance metric, which measures the distance between data items, and clusters together data items that are closer to each other. Clustering techniques include the MacQueen's K-means algorithm and the Kohonen's Self-Organizing Map algorithm.

Learning algorithms asserted for use in classifying biological information are described, for example, in PCT International Publication No. WO 01/31580 (Barnhill et al., “Methods and Devices for Identifying Patterns in Biological Systems and Methods of Use Thereof”), U.S. patent application No. 2002/0193950 A1 (Gavin et al., “Method or analyzing mass spectra”), U.S. patent application No. 2003/0004402 A1 (Hitt et al., “Process for Discriminating Between Biological States Based on Hidden Patterns from Biological Data”), and U.S. patent application No. 2003/0055615 A1 (Zhang et al., “Systems and Methods for Processing Biological Expression Data”).

The classification models can be formed on and used on any suitable digital computer. Suitable digital computers include micro, mini, or large computers using any standard or specialized operating system, such as a Unix, Windows™, or Linux™, based operating system. The digital computer that is used may be physically separate from the device that is used to create the data of interest, or it may be coupled to such device.

The training data set and the classification models according to embodiments of the invention can be embodied by computer code that is executed or used by a digital computer. The computer code can be stored on any suitable computer readable media including optical or magnetic disks, sticks, tapes, etc., and can be written in any suitable computer programming language including C, C++, visual basic, etc.

The learning algorithms described above are useful both for developing classification algorithms for the biomarkers already discovered, or for finding new biomarkers for ovarian cancer. The classification algorithms, in turn, form the base for diagnostic tests by providing diagnostic values (e.g., cut-off points) for biomarkers used singly or in combination. In one embodiment, the classification algorithm is the product of a learning algorithm. In a related embodiment, the learning algorithm is trained on biomarker levels from known malignant ovarian cancer samples.

In the case of complex classification algorithms, it may be necessary to perform the algorithm on the data, thereby determining the classification, using a computer, e.g., a programmable digital computer. In either case, one can then record the status on tangible medium, for example, in computer-readable format such as a memory drive or disk or simply printed on paper. The result also could be reported on a computer screen.

In one embodiment, classification algorithms are used to determine a cutoff value from the measured amounts of biomarkers selected from the group consisting of transthyretin, apolipoprotein A1, transferrin, β-2 microglobulin, and CA 125 II. In another embodiment, the classification algorithm is a linear regression formula.

Diagnosis of Ovarian Cancer

Methods described herein are useful for the diagnosis of ovarian cancer. They can also be combined with or supplement traditional methods used to diagnose ovarian cancer. Other methods include a physical examination (including a pelvic examination), a blood test (for various biomarkers), and transvaginal ultrasound. The diagnosis is traditionally confirmed with surgery to inspect the abdominal cavity, take biopsies (tissue samples for microscopic analysis) and look for cancer cells in the abdominal fluid.

Ovarian cancer at its early stages (I/II) is difficult to diagnose until it spreads and advances to later stages (III/IV). This is because most symptoms are non-specific and thus of little use in diagnosis. The serum BHCG level is typically measured in any female in whom pregnancy is a possibility. In addition, serum alpha-fetoprotein (AFP) and lactate dehydrogenase (LDH) is typically measured in young girls and adolescents with suspected ovarian tumors because the younger the patient, the greater the likelihood of a malignant germ cell tumor.

The OVA1 FDA-approved test (available commercially from Quest Diagnostics, Inc.) tests for the biomarkers transthyretin, apolipoprotein A1, transferrin, β-2 microglobulin, and CA 125 II and uses an algorithm to indicate the probability of malignancy of an ovarian mass based on the test results of these five biomarkers. It is not a screening or standalone test but when used in conjunction with a standard pre-surgical evaluation this test can be used to:

assess the likelihood that an ovarian mass is malignant before its removed;

help to identify patients for referral to a gynecologic oncologist and

may produce improved patient outcomes.

In the presence of ovarian serous carcinoma, CA 125 II will increase as will beta 2 microglobulin Apolipoprotein A1, pre-albumin (transthyretin) and transferrin will decrease. In large increases of CA125 II, the score will be weighted in favor of showing a high risk of malignancy. Other carcinomas of ovarian origin or metastatic origin will likely increase the OVA1 score to above the cutoff levels.

A pelvic examination and imaging including CT scan and trans-vaginal ultrasound are essential. Physical examination may reveal increased abdominal girth and/or ascites (fluid within the abdominal cavity). Pelvic examination may reveal an ovarian or abdominal mass. The pelvic examination can include a rectovaginal component for better palpation of the ovaries. For very young patients, magnetic resonance imaging may be preferred to rectal and vaginal examination.

To definitively diagnose ovarian cancer, a surgical procedure to take a look into the abdomen is usually performed. This can be an open procedure (laparotomy, incision through the abdominal wall) or keyhole surgery (laparoscopy). During this procedure, suspicious areas will be removed and sent for microscopic analysis. Fluid from the abdominal cavity can also be analysed for cancerous cells. If there is cancer, this procedure can also determine its spread (which is a form of tumor staging).

### EXAMPLE 1

### IL-6 High Sensitivity ELISA

This assay employs the quantitative sandwich enzyme immunoassay technique. The wells of a microplate are pre coated with an IL-6 specific monoclonal antibody. When pipetted into the wells, the IL-6 present in any of the standards, controls, and samples is immobilized by the monoclonal antibody. After washing away any unbound substances, an enzyme linked polyclonal antibody specific to IL-6 is added to the wells. Following a wash to remove any unbound enzyme-antibody, a substrate solution is added to the wells. After an incubation period, an amplifier solution is added to develop a colored signal. The intensity of the color, which is proportional to the amount of IL-6 bound in the initial step, is quantified by a plate reader.

Specimen Requirements. Cytokine levels may demonstrate diurnal variation. Recommend cytokine levels be determined at the same time of day for improved longitudinal comparison.

Specimen Type & Handling. Specimen types useful in this IL-6 ELISA include but are not limited to Serum, Plasma, Plasma with added EDTA, human breast milk, and vaginal swabs. About 1 mL of a sample is collected from the subject and either analyzed or frozen for future analysis.

Reagent preparation. A microtest strip (96 well polystyrene microplate coated with mouse monoclonal antibody against IL-6) is allowed to equilibrate to room temperature (18-26° C.). Next, the wash buffer is prepared. Concentrated wash buffer solution (100 mL of 10× solution of buffered surfactant with preservatives) is warmed to room temperature and mixed gently to allow for the crystals to dissolve. lx wash buffer is made by mixing 100 mL of the 10× solution with 900 mL DI-water (Nerl or equivalent). The 10 pg/ml standard is prepared by adding 5 mL of RD6-11 (21 mL of a buffered protein base with preservatives) to lyophilized IL-6 (50 pg lyophilized recombinant human IL-6) at least 15 minutes prior to use. The standard is allowed to sit for a minimum of 15 minutes with gentle agitation prior to making dilutions. The substrate is prepared by reconstituting lyophilized substrate (lyophilized NADPH with stabilizers) with 6 mL of Substrate Diluent (7 mL of buffered solution with stabilizers) at least 10 minutes before use. The substrate is capped and thoroughly mixed. The amplifier solution is prepared by reconstituting lyophilized Amplifier (Lyophilized amplifier enzymes with stabilizers) with 6 mL of Amplifier Diluent (7 mL of buffered solution containing INT-violet with stabilizers) at least 10 minutes before use. The vial is capped and mixed thoroughly. Human IL-6 at high, medium, and low concentrations is used as quality control standards.

Preparation of IL-6 Standard Curve. The following concentrations of standard are prepared from the 10 pg/ml standard: 0.0 pg/mL (calibrator diluent RD6-11 only), 0.156 pg/mL, 0.312 pg/mL, 0.625 pg/mL, 1.25 pg/mL, 2.5 pg/mL, 5.0 pg/mL, and 10 pg/mL. The eight standards are run in duplicate with every assay set-up. Up to 4 standard curve singlicate ODs (or 2 non-consecutive standard points) can be rejected if they exceed 20% CV.

Assay. Patient samples and controls are thawed at room temperature and samples are mixed. For breast milk samples preparation, only the aqueous fraction of the breast milk is needed for testing. To obtain aqueous fraction, breast milk samples are centrifuged at 700 to 720g for 20 min at room temperature and then incubated at 2-8° C. for 5 minutes without disturbing the fatty layer. After 5 minutes in the refrigerator, using disposable Pasteur pipettes the aqueous fraction is transferred to another tube without disturbing the fatty layer. For vaginal swabs sample, the sample is mixed and the swab is removed before pipetting.

The microtiter plate is set up with sufficient wells for running standards and controls in duplicate. 100 μL of the Assay Diluent RD1-75 (11 mL of a buffered protein base with preservatives) is added to each well of the microtiter plate. 100 μL of each standard, control, and patient sample (initial testing for sample is run undiluted) is added into the appropriate wells. Patient samples can be diluted according to the following Table:

| | Patient Sample | Calibrator Diluent |
|---|---|---|
| Dilution | in μL | RD6-11 in μL |
| 1:2 | 200 | 200 |
| 1:8 | 100 of 1:2 | 300 |
| 1:32 | 100 of 1:8 dilution | 300 |
| 1:256 | 100 of 1:32 dilution | 700 |
| 1:1280 | 100 of 1:256 | 400 |

The plate is covered with the plate sealer and incubated at room temperature on a plate shaker at 500±50 rpm for 120 minutes. Next, the plate is washed 6 times, turned upside down and tapped on towels to remove any of the remaining wash buffer. 200 μL of Conjugate (21 mL of polyclonal antibody against IL-6, conjugated to alkaline phosphatase, with preservatives) is added to each well. The plate is then covered and incubated at room temperature on a plate shaker at 500±50 rpm for 120 minutes. Next, the plate is washed 6 times 50 μL of substrate is added to each well. The plate is then covered and incubated at room temperature for 60 minutes. Next, 50 μL of amplifier solution is added to each well and the plate is covered and incubated at room temperature for 30 minutes. Next, 50 μL of stop solution (6 mL of 2N sulfuric acid) is added to each well. Plate is then read on Powerwave Plate Reader set at 490 nm with corrections at 650 nm.

Calculations. The average absorbance values for each set of duplicate standards, controls, and samples is calculated. The calibration curve is obtained by plotting the standards' concentrations in pg/mL versus the corresponding A₄₉₀₋₆₅₀. A log/log linear curve fit subtracting the zero standard (blank) is used. Samples with a concentration above or equal to the second highest standard and up to the highest standard will be diluted with Calibrator Diluent RD6-11 and re-tested at NT, 1:8, 1:32, 1:256 or higher until the OD's are within the standard curve. The test sample is fit on the curve and the concentration of IL-6 in the test sample is determined.

Interleukin 6 (IL-6) may be considered the protypical pleiotrophic cytokine Human IL-6 is a variably glycosylated 22-27 kDa glycoprotein. IL-6 is translated as a 212 amino acid (aa) molecule, which incorporated a 28 aa signal and a 184 aa mature segment. Expression of IL-6 has been observed in CD8 +T cells, fibroblasts, synoviocytes, adipocytes, osteoblasts, megakaryocytes, endothelial cells, sympathetic neurons, cerebral cortex neurons, adrenal medulla chromaffin cells, retinal pigment cells, mast cells, keratinocytes, Langerhans cells, fetal and adult astrocytes, neutrophils, monocytes, eosinophils, colonic epithelial cells, B1 B cells, and most likely pancreatic islet beta cells. The production of IL-6 is generally correlated to cell activation. IL-6 has been described as both pro- and anti-inflammatory molecule, a modulator of bone resorption, a promoter of hematopoiesis, and an inducer of plasma cell development. In normal individuals the circulating IL-6 found in the blood is in the range of 1 pg/mL, with slight elevations during the menstrual cycle, modest elevations during some cancers, and large elevations following surgery.

### EXAMPLE 2

### Determining Specificity and Sensitivity of Tests for Ovarian Cancer

Using assays and methods described herein, the amount of IL-6 was determined in samples with known ovarian cancer status. The OVA-1 score was also determined in the same samples. The OVA-1 is a commercially available test (Vermillion, Inc.) described previously. The specificity and sensitivity of each test was calculated as follows: Sensitivity=True Positives/(True Postives+False Negatives); Specificity=True Negatives/(True Negatives+False Positives).

The results of these tests and specificity and sensitivity are tabulated below:

| | | IL-6 | |
|---|---|---|---|
| Patients | OVA-1 | (pg/mL) | Diagnosis |
| Pooled #1 | 2.3 | 2.7 | Benign |
| Pooled #2 | 1.9 | 3.4 | Benign |
| Pooled #3 | 2.1 | 3.7 | Benign |
| Pooled #4 | 1.7 | 4.7 | Benign |
| Pooled #5 | 5.3 | 9.9 | Malignant |
| Patient 65035811 | 9.7 | 1042.0 | Malignant (“Ovarian Malignancy |
| | | | with positive nodes) |
| Patient 65005473 | 8.0 | 1.8 | Benign (“Endometrioma of the |
| | | | ovary”) |
| Patient 65014335 | 6.9 | 40.7 | Malignant (“Ovarian cancer and |
| | | | renal cancer”) |
| Patient 65069832 | 6.3 | 5.0 | Benign (“Hydrosalpinx”) |
| Patient 65087301 | 8.2 | 3.4 | (“Metastatic esophageal |
| | | | adenocarcinoma”) |
| Patient 65061954 | 8.2 | 5.0 | Benign (“Cystadenoma of the |
| | | | ovary”) |
| Patient 65062186 | 3.5 | 2.0 | Benign (“Benign hemorrhagic |
| | | | ovarian cyst”) |
| Patient 65041032 | 7.4 | 8.7 | Malignant |
| | | | (“Anaplastic carcinoma”) |
| Patient 65050870 | 9.1 | 31.4 | Malignant (“Carcinomatosis”) |
| Patient 65047450 | 6.6 | 2.8 | Benign (“No adnexal mass; benign |
| | | | endometrial biopsy”) |
| Patient 65039328 | 5.1 | 0.9 | Benign (“Benign ovarian cyst”) |
| Patient 65005240 | 7.9 | 7.9 | Benign (“No ovarian mass; |
| | | | adenomyosis”) |
| Patient 65074605 | 6.3 | 2.3 | Benign (“Clinically benign and lost |
| | | | to followup”) |
| Sensitivity | 100% | 83% | |
| Specificity | 42% | 92% | |

OVA-1 cutoffs are: pre-menopausal is about 4.4 and postmenopausal is about 5.0. IL-6 cutoff is about 5.00 pg/mL.

Using the same OVA-1 scores and IL-6 amounts, the specificity and sensitivity was determined with an OVA-1 score greater than 8.1 in the presence of a normal IL6 (i.e. less than or equal to 5.0 pg/mL) and with an OVA1-score greater than the current cutoffs (i.e. 4.4/5.0) in the presence of an elevated IL6 (i.e. greater than 5.0 pg/mL). These criteria would produce a sensitivity of 100% and specificity of 89%.

The contents of the articles, patents, and patent applications, and all other documents and electronically available information mentioned or cited herein, are hereby incorporated by reference in their entirety to the same extent as if each individual publication was specifically and individually indicated to be incorporated by reference. Applicants reserve the right to physically incorporate into this application any and all materials and information from any such articles, patents, patent applications, or other physical and electronic documents.

The inventions illustratively described herein may suitably be practiced in the absence of any element or elements, limitation or limitations, not specifically disclosed herein. Thus, for example, the terms “comprising”, “including,” “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the inventions embodied therein herein disclosed may be resorted to by those skilled in the art, and that such modifications and variations are considered to be within the scope of this invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims. In addition, where features or aspects of the invention are described in terms of Markush groups, those skilled in the art will recognize that the invention is also thereby described in terms of any individual member or subgroup of members of the Markush group.

That which is claimed is:

1. A method for identifying the risk of an individual for having ovarian cancer comprising:
(a) measuring, in a biological sample from the subject, the amount of IL-6;
(b) comparing the amount of IL-6 measured in step (a) to a predetermined IL-6 cutoff value; and
(c) identifying the individual as being at risk for having ovarian cancer when the amount of IL-6 is greater than the IL-6 cutoff value, and identifying the individual as not at risk for having ovarian cancer when the amount of IL-6 is less than the IL-6 cutoff value.

2. The method of claim 1, wherein the predetermined cutoff value is derived from a measurement of the amount of IL-6 in one or more subjects that do not have ovarian cancer.

3. The method of claim 1, wherein the biological sample is serum or plasma.

4. The method of claim 3, wherein the IL-6 cutoff value is about 5 pg/ml.

5. The method of claim 1, further comprising
(i) measuring, in the biological sample, the amount of a biomarker selected from the group consisting of transthyretin, apolipoprotein Al, transferrin, β-2 microglobulin, and CA 125 II,
(ii) comparing the amount of the biomarker measured in step (i) to a predetermined biomarker cutoff value; and
(iii) identifying the individual as being at risk for having ovarian cancer when the amount of IL-6 is greater than the IL-6 cutoff value and the amount of the biomarker is greater than the biomarker cutoff value, and identifying the individual as not at risk for having ovarian cancer when either or both of the amount of IL-6 is less than the IL-6 cutoff value and the amount of the biomarker is less than the biomarker cutoff value.

6. The method of claim 1, further comprising
(i) measuring, in the biological sample, the amount of two or more biomarkers selected from the group consisting of transthyretin, apolipoprotein A1, transferrin, β-2 microglobulin, and CA 125 II,
(ii) calculating a biomarker score from the results of step (i);
(iii) comparing the biomarker score to a predetermined biomarker score cutoff value; and
(iv) identifying the individual as being at risk for having ovarian cancer when the amount of IL-6 is greater than the IL-6 cutoff value and the biomarker score is greater than the biomarker score cutoff value, and identifying the individual as not at risk for having ovarian cancer when either or both of the amount of IL-6 is less than the IL-6 cutoff value and the biomarker score is less than the biomarker score cutoff value.

7. The method of claim 6, wherein the amount of three or more biomarkers selected from the group consisting of transthyretin, apolipoprotein A1, transferrin, β-2 microglobulin, and CA 125 II are measured and used to calculate the biomarker score.

8. The method of claim 6, wherein the amount of four or more biomarkers selected from the group consisting of transthyretin, apolipoprotein A1, transferrin, β-2 microglobulin, and CA 125 II are measured and used to calculate the biomarker score.

9. The method of claim 6, wherein the amount of transthyretin, apolipoprotein A1, transferrin, β-2 microglobulin, and CA 125 II are measured and used to calculate the biomarker score.

10. The method of any one of claims 1-9, wherein the subject is a post-menopausal woman.

11. The method of any one of claims 1-9, wherein the subject is a pre-menopausal woman.

12. The method of any one of claims 1-11, wherein the cutoff value is determined by a classification algorithm.

13. The method of claim 12, wherein the classification algorithm is a linear regression formula.

14. The method of claim 13, wherein the classification algorithm is the product of a learning algorithm.

15. The method of claim 14, wherein the learning algorithm is trained on biomarker levels from known malignant ovarian cancer samples.

16. The method of any one of claims 1-15, wherein the amount of IL-6 and the one or more biomarkers are measured by one or more method selected from the group consisting of immunonephelometry, electrochemiluminescence, and ELISA.

17. The method of claim 16 wherein the IL-6 is measured by ELISA.

18. The method of claim 16 or 17 wherein transthyretin, apolipoprotein A1, β-2 microglobulin, and transferrin are measured by the method of immunonephelometry.

19. The method of any one of claims 16-18 wherein CA 125 II is measured by the method of electrochemiluminescence.
